(19) United States
(12) Patent Application Publication
(10) Pub. No.: US 2021/0220181 A1
(43) Pub. Date: Jul. 22, 2021

(54) HYDROPOLYMER FOAM WITH ADDITIONAL COMPONENT TO INCREASE ABSORBENT CAPACITY

(71) Applicants: SYSTAGENIX WOUND MANAGEMENT, LIMITED, West Sussex (GB); KCI USA, INC., San Antonio, TX (US)

(72) Inventors: Leanna J. POOLE, Rawtenstall (GB); Clinton R. HILL, Colne (GB); Rachel E. BOLTON, Halifax (GB)

(21) Appl. No.: 16/760,596

(22) PCT Filed: Oct. 31, 2018
(86) PCT No.: PCT/US2018/058497

§ 371 Date: Apr. 30, 2020

Related U.S. Application Data
(60) Provisional application No. 62/580,865, filed on Nov. 2, 2017.

(51) Int. Cl. A61F 13/02; A61F 13/00; A61L 15/42; A61L 15/40; A61L 15/60

(52) U.S. Cl. CPC A61F 13/0213; A61F 13/00063; A61L 15/425; A61F 2013/00927; A61L 15/60; A61F 2013/0074; A61F 2013/00748; A61L 15/40

(57) ABSTRACT

The present disclosure describes a wound dressing that includes one or more antioxidants in the dressings absorbent layer. In some implementations, the antioxidant includes green tea. The antioxidant containing wound dressing can both increase the absorbency of the dressings foam and increase the cellular proliferation of the wound. With increased absorbency, the dressing can handle higher amounts of exudate compared to a hydropolymer foam without the green tea (or other antioxidant) addition. The cell proliferation component of the wound dressing described herein can decrease a wounds healing time, by increasing the cells proliferation and hence regenerating healthy skin faster.

### CROSS-REFERENCE TO RELATED APPLICATIONS

This application claims the benefit of and priority to U.S. Provisional patent Application No. 62/580,865, filed Nov. 2, 2017, the entire contents of which are incorporated herein by reference.

### BACKGROUND OF THE DISCLOSURE

Maintaining a moist wound environment can promote the healing of wounds, especially burns and chronic wounds such as ulcers. Wound fluid is produced by the wound during the healing process. Wound fluid is mainly composed of water, but also contains nutritional requirements, proteins, and cells. Wound fluid represents an essential component of the healing process. However, in the case of chronic wounds prolonged inflammation and infection can lead to excess and prolonged production of exudate. Absorbent dressings are designed to manage the excess exudate produced. Absorbent wound dressings can have a maximum fluid handling capacity. Once breached, the dressing can fail and the wound fluid can escape the confines of the wound area. This can cause patient discomfort and can cause maceration of skin adjacent to the wound. Predicting or otherwise determining when a wound dressing has reached its maximum fluid handling capacity can be difficult.

### SUMMARY OF THE DISCLOSURE

According to an aspect of the disclosure, a wound dressing can include a backing layer that can have a first environment-facing side and a first wound-facing side. The backing layer can be liquid impermeable and vapor permeable. The wound dressing can include a wicking layer that can have a second environment-facing side and a second wound-facing side. The second environment-facing side of the wicking layer can be coupled with the first wound-facing side of the backing layer. The wound dressing can include an absorbent layer that can include between about 1% and about 10% green tea by weight. The absorbent layer can have a third wound-facing side and a third environment-facing side. The third environment-facing side of the absorbent layer can be coupled with the second wound-facing side of the absorbent layer.

In some implementations, the absorbent layer can include between about 1% and about 5% green tea by weight. The absorbent layer can include a hydropolymer. The absorbent layer can include between 10% and 90% polyurethane by weight.

In some implementations, the green tea is configured to increase a pore size of a plurality of pores in the absorbent layer. The green tea can be configured to increase an absorbency of the absorbent layer. The green tea can be configured to increase a rate of cellular proliferation. In some implementations, the wicking layer can include rayon.

According to an aspect of the disclosure, a method to form a wound dressing can include providing a backing layer. The backing layer can include a first environment-facing side and a first wound-facing side. The backing layer can be liquid impermeable and vapor permeable. The method can include coupling a second environment-facing side of a wicking layer with the first wound-facing side of the backing layer. The method can include coupling a third environment-facing side of an absorbent layer with the second wound-facing side of the wicking layer. The absorbent layer can include between about 1% and about 10% green tea by weight.

In some implementations, the absorbent layer can include between about 1% and about 5% green tea by weight. The absorbent layer can include a hydropolymer. The absorbent layer can include between 10% and 90% polyurethane by weight. The green tea can increase a pore size of a plurality of pores in the absorbent layer. The green tea can increase an absorbency of the absorbent layer. The green tea can increase a rate of cellular proliferation. The wicking layer can include rayon.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates a top view an example wound dressing.

FIG. 2 illustrates an exploded view of the example wound dressing illustrated in FIG. 1.

FIG. 3 illustrates a cross-sectional view of the example wound dressing illustrated in FIG. 1.

FIG. 4 illustrates a plot 400 of the levels of cell proliferation using the wound dressing illustrated in FIG. 1 and other wound dressings.

FIGS. 5A-5C illustrate scanning electron microscopy images of example absorbent layers that can be used in the wound dressing 100 illustrated in FIG. 1.

FIG. 6 illustrates an example method to form the wound dressing 110 illustrated in FIG. 1.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure describes a wound dressing that includes one or more antioxidants in the dressing's absorbent layer. In some implementations, the antioxidant includes green tea. The antioxidant containing wound dressing can both increase the absorbency of the dressing's foam and increase the cellular proliferation of the wound. With increased absorbency, the dressing can handle higher amounts of exudate compared to a hydropolymer foam without the green tea (or other antioxidant) addition. The cell proliferation component of the wound dressing described herein can decrease a wounds healing time, by increasing the cells proliferation and hence regenerating healthy skin faster.

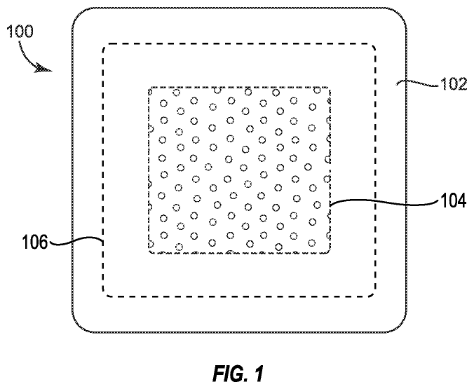

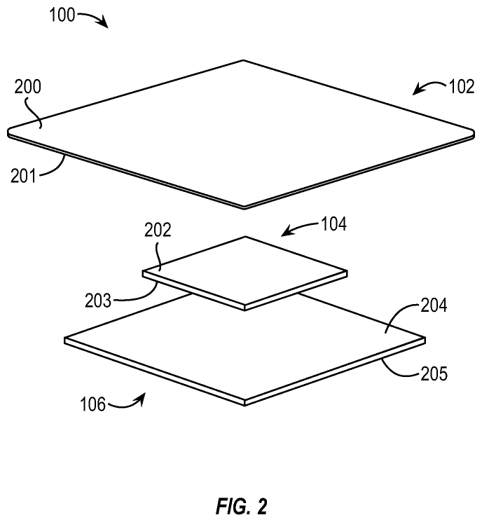

FIG. 1 illustrates a top view an example wound dressing 100. FIG. 2 illustrates an exploded view of the example wound dressing illustrated in FIG. 1. Referring to FIGS. 1 and 2 together, the wound dressing 100 includes a top, backing layer 102. Beneath the backing layer 102, as indicated by the dotted lines, the wound dressing 100 includes a wicking layer 104 and an absorbent layer 106. The absorbent layer 106 can include green tea or other antioxidants. The green tea or other antioxidants can increase the pore size of the absorbent layer 106 and increase the absorbent layer's absorbency. The wound dressing 110 can include a wicking layer 104. In some implementations, the wound dressing 110 does not include a wicking layer 104. The wicking layer 104 can distribute fluid across the wound dressing 110 and enable moisture vapor transfer. For example, the wicking layer 104 can facilitate the distribution of fluid through the absorbent layer 106. The wicking layer 104 can include a non-woven material.

The wicking layer 104 can be made of any material suitable for providing a wicking function to enable fluid transfer between different areas of the absorbent layer 106. In some implementations, the wicking layer 104 can include rayon or other suitable wicking fabric. Several examples of wicking materials that can be used in wicking layer 104 are described in detail in U.S. patent application Ser. No. 13/009,238 filed Jan. 19, 2011, U.S. Pat. No. 6,936,037 filed Apr. 8, 2003, and U.S. Pat. No. 9,456,930 filed Mar. 17, 2014. The entire disclosure of each of these patents and patent applications is incorporated by reference herein. In some implementations, the wicking properties of the wicking layer 104 can be enhanced by using a hydrophilic material.

The wound dressing's backing layer 102 is a liquid impermeable and vapor permeable barrier. The backing layer 102 can be impermeable to wound exudate or other wound fluids. The backing layer 102 provides a barrier to the passage of microorganisms, bacteria, and other contaminants through the wound dressing 100. The backing layer 102 enables the other components of the wound dressing 100 to retain fluid to promote a moist wound environment.

The backing layer 102 can be a thin layer of polyurethane film. For example, the backing layer 102 can include the polyurethane film ESTANE 5714F. The backing layer 102 can include poly alkoxyalkyl acrylates and methacrylates. In some implementations, the backing layer 102 includes a continuous layer of a high-density blocked polyurethane foam that is predominantly closed-cell.

The backing layer 102 extends beyond the perimeter of the wicking layer 104 and the absorbent layer 106. The backing layer's wound-facing side can be coated with an acrylic or other adhesive. The adhesive couples with the wicking layer 104 and the absorbent layer 106. The portion of the backing layer 102 extending past the absorbent layer 106 adheres to the patient or other surface during wear time. The backing layer's adhesive-coated margin that extends past the absorbent layer 106 can extend past all sides of the absorbent layer 106 such that wound dressing 100 is a so-called island dressing. In other implementations, the adhesive-coated margin can be eliminated and wound dressing 100 can be adhered to a surface using other techniques.

The adhesive applied to the wound-facing side of the backing layer 102 can be moisture vapor transmitting. The application of the adhesive can be patterned to enable the passage of water vapor through the backing layer 102. The adhesive may include a continuous moisture vapor transmitting, pressure-sensitive adhesive layer of the type, conventionally used for island-type wound dressings (e.g., a polyurethane-based pressure sensitive adhesive). One example of an adhesive which can be used is a pressure sensitive adhesive based on acrylate ester copolymers, polyvinyl ethyl ether and polyurethane.

The wound dressing 100 also includes the absorbent layer 106. The absorbent layer 106 can include one or more antioxidants. In some implementations, the antioxidant is green tea. The absorbent layer 106 can include between about 1% and about 10%, between about 1% and about 8%, between about 1% and about 5%, between about 1% and about 4%, between about 1% and about 3%, or between about 1% and about 2% green tea by weight.

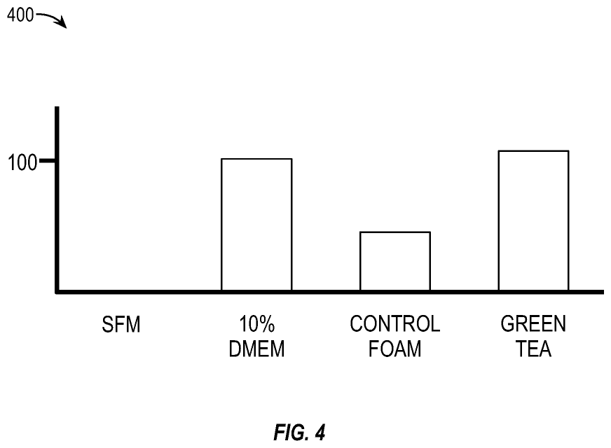

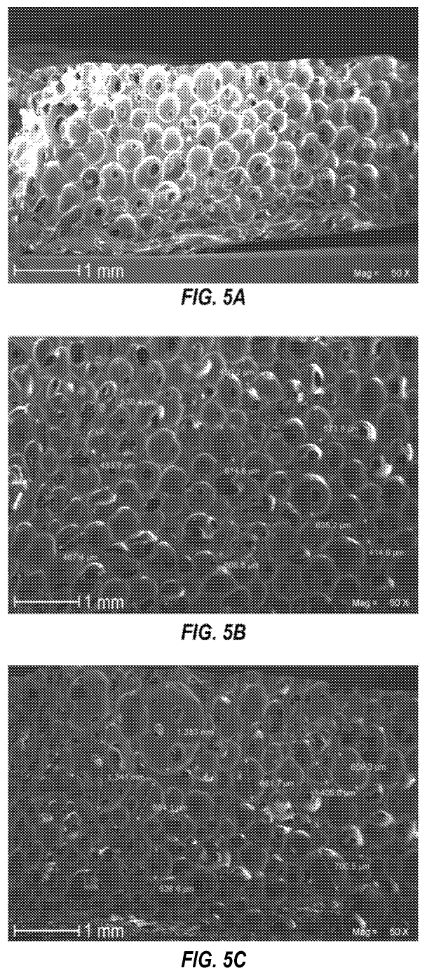

In some implementations, as described further in relation to FIG. 4, the addition of green tea to the absorbent layer 106 can increase cell proliferation in the wound to which the wound dressing 110 is applied. In some implementations, as described further in relation to FIGS. 5A-5C, the green tea (or other antioxidant) can increase the absorbency of the absorbent layer 106. The green tea can increase the absorbency by increasing the pore size of the pores in the absorbent layer 106. The increased pore size can enable the absorbent layer 106 to retain more fluid when compared to an absorbent layer 106 with relatively smaller pores. The green tea can increase the absorbency of the absorbent layer 106 without altering the volume or shape of the absorbent layer 106. For example, an absorbent layer 106 of a given volume that includes green tea can absorb more fluid when compared to an absorbent layer 106 of the save volume without green tea. The addition of green tea to the absorbent layer 106 can also enable greater absorbency without affecting the compliance of the absorbent layer 106 (e.g., the absorbent layer, with and without green tea, has substantially the same level of flexibility).

The wound dressing 100 also includes the absorbent layer 106. The absorbent layer 106 can be a hydrophilic foam layer. The absorbent layer 106 may include a polyurethane foam. In some implementations, the absorbent layer 106 includes a flexible plasticized hydrophilic polymer matrix having an internal cellular structure. Several examples of hydrophilic foams which can be used to form the absorbent layer 106 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein. Green tea or other antioxidants can be added to any of the above-described foams.

The absorbent layer 106 can provide enhanced absorbency for liquid exudate. This is because the initial substantially anhydrous condition and porous structure of the absorbent layer 106 enables it to absorb a larger amount of water by both chemical and physical absorption that is the case for the corresponding hydrogel material. Furthermore, the porous structure of the foam provides for rapid uptake of liquid exudate, in contrast to pure hydrogel dressings.

In some implementations, the absorbent layer 106 can include a hydrogel or and a hydrogel composition that can include any hydrophilic gels and gel compositions. The compositions can include organic non-polymeric components in the absence of water. For example, the absorbent layer 106 can be formed from a polyurethane foam that entraps water to form a gel. The absorbent layer 106 can be substantially continuous, substantially non-porous, or non-foamed. The absorbent layer 106 can include a flexible plasticized hydrophilic polymer matrix having a substantially continuous internal structure. The density of absorbent layer 106 may be between about 0.5 g/cm\hat{ }3 and about 1.1 g/cm\hat{ }3, between about 0.8 g/cm\hat{ }3 and about 1.1 g/cm\hat{ }3, or between about 0.9 and about 1.1 g/cm\hat{ }3.

In some implementations, the absorbent layer 106 can include a hydropolymer formed from at least one of vinyl esters, vinyl ethers, carboxy vinyl monomers, meth(acrylic) acid, acrylamide, N-vinyl pyrrolidone, acylamidopropanem acylamidopropane, PLURONIC (Maleic acid, NN-dimethylacrylamide diacetone acrylamide acryloyl), morpholine, polyurethane and mixtures thereof.

In some implementations, the absorbent layer 106 is cross-linked. The absorbent layer 106 can be substantially insoluble in water at ambient temperatures. The absorbent layer 106 can absorb and entrap liquid to provide a highly hydrated gel structure. The gel of the absorbent layer 106 can absorb between about 1 g/g and about 10 g/g, between about 2 g/g and about 7 g/g, or between about 2 g/g and about 5 g/g of physiological saline at 20°.

In some implementations, the absorbent layer 106 has an absorbency of between about 2 g and about 6 g, between about 3 g and about 6 g, or between about 4 g and about 6 g of saline per gram of foam. The absorbent layer 106 can have a swellability in water of at least 200%. In some implementations, the absorbent layer 106 includes less than 10%, less than 5%, or less than 2% of water before use.

In some implementations, the dry weight of the absorbent layer 106 is from about 1000 g/m\hat{ }2 to about 5000 g/m\hat{ }2 or between about 2000 g/m\hat{ }2 to about 4000 g/m\hat{ }2. In some implementations, the absorbent layer 106 includes between about 1% and about 30%, between about 5% and about 25%, or between about 10% and about 20% by weight of water before use. The absorbent layer 106 can contain between about 1% and about 40%, between about 5% and about 20%, or between about 5% and about 15% by weight one or more humectants. The humectants can include glycerol, propylene glycol, sorbitol, mannitol, polydextrose, sodium pyrrolidine carboxylic acid (NaPCA), hyaluronic acid, aloe, jojoba, lactic acid, urea, gelatin, lecithin, or any combination thereof. The entrapped water and optional humectants can give the hydrogel a soft, moist wound-friendly surface for contacting the wound.

FIG. 2 illustrates an exploded view of an example wound dressing 100. The wicking layer 104 is positioned between the absorbent layer 106 and the backing layer 102. The backing layer 102 includes an environment-facing side 200 and a wound-facing side 201. The wicking layer 104 includes an environment-facing side 202 and a wound-facing side 203. The absorbent layer 106 includes an environment-facing side 204 and a wound-facing side 205.

The backing layer 102 includes the environment-facing side 200 and the wound-facing side 201. The wound-facing side 201 can be coated with an adhesive. The adhesive can coat the wound-facing side 201 or can be patterned onto the wound-facing side 201. For example, the adhesive can be patterned on the margin of the wound-facing side 201 that is beyond the perimeter of the absorbent layer 106 and ultimately contacts that patient's skin.

The wound-facing side 201 of the backing layer 102 is coupled with the environment-facing side 202 of the wicking layer 104. In some implementations, an adhesive can be used to couple the backing layer 102 and the wicking layer 104. In other implementations, the coupling of the absorbent layer 106 and the backing layer 102 can seal the wicking layer 104 between the absorbent layer 106 and the backing layer 102 and hold the wicking layer 104 in position. In some implementations, the absorbent layer 106 and the wicking layer 104 (the “island materials”) are coupled to the backing layer 102 by a wound contact layer. For example, the wound contact layer is a perforated sheet (such as polyurethane film) coated on the wound-facing side with silicone, and the environment-facing side with a pressure-sensitive adhesive (such as acrylic adhesive). The wound contact layer is the same or substantially the same size as the backing layer 102, and is affixed to backing layer 102 via the pressure-sensitive adhesive, resulting in the island material being held in place between the wound contact layer and backing 102 (without the need for an adhesive between the island material and the backing layer 102).

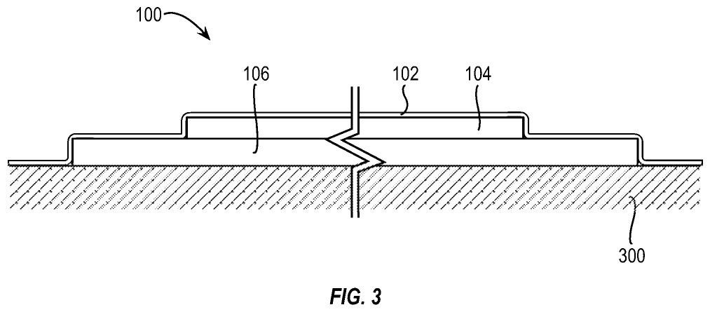

FIG. 3 illustrates a cross-section of the wound dressing 110. As described above, the backing layer 102 can extend past the perimeter of the wicking layer 104 and the absorbent layer 106. The portion of the backing layer 102 extending past the absorbent layer 106 can coupled the wound dressing 110 with the patient 300 (or other surface).

In some implementations, the backing layer 102 can have a thickness between about 10 μm and about 100 μm, between about 25 μm and about 75 μm, or between about 50 μm and about 75 μm. The thickness of the absorbent layer 106 can be between about 1 mm and about 10 mm, between about 2 mm and about 7 mm, or between about 2 mm and about 5 mm.

FIG. 4 illustrates a plot 400 of the levels of cell proliferation using the wound dressing illustrated in FIG. 1 and other wound dressings. A wound that has increased cellular proliferation is also more likely to heal quicker than a wound without increased cellular proliferation, which reduces healing time.

For each of the foam materials, portions of foam containing green tea or control foam (e.g., foam without green tea) were placed into wells. Control wells included only serum-free media (SFM) or 10% Dulbecco's modified eagle medium (DMEM). Cells were then seeded into each of the wells at a concentration of 4×10³ cells/well. The cells were then cultured for 4 days. After 4 days, a labeling mixture was added to each of the wells and then the wells were incubated for another 4 days. The cellular proliferation was tested by measuring the spectrophotometrical absorbance of the samples using a microplate (ELISA) reader.

In the cell proliferation assay a difference of P=0.05, was seen between the control foam and the green tea foam. Although a wide standard deviation was seen there was no significant difference shown between 10% DMEM and the green tea foam, showing a high percentage of cell growth from the green tea foam.

FIGS. 5A-5C illustrate scanning electron microscopy (SEM) images of example absorbent layers that can be used in the wound dressing 100 illustrated in FIG. 1. FIG. 5A illustrates a SEM image of the absorbent layer with no green tea added to the absorbent layer. FIG. 5B illustrates a SEM image where 0.5 grams of green tea (˜1% green tea by weight) were added to the absorbent layer. FIG. 5C illustrates an absorbent layer where 2 grams of green tea (˜4% green tea by weight) were added to the absorbent layer. As illustrated in FIGS. 5A-5C, the pore size within the absorbent layers shows that the pore size of the absorbent layer's foam increases as the volume of the green tea increases. The increase in pore size results in an increase in overall absorbency. Table 1 illustrates the results of an absorbency test with the absorbent layers illustrated in FIGS. 5A-5C. Table 1 and FIGS. 5A-5C illustrate that the foam that included 2 g of green tea have a higher absorbency and the greatest pore size compared to the control which contained no green tea.

| | | Dry Weight | Wet Weight | Absorbency | Mean |
|---|---|---|---|---|---|
| Sample | Description | (g) | (g) | (g/g) | (g/g) |
| 1 | Control Foam | 0.92 | 10.48 | 10.39 | 10.23 |
| 2 | Control Foam | 1.01 | 11.18 | 10.07 | |
| 3 | 2 g Green Tea Foam | 0.91 | 12.71 | 12.97 | 13.56 |
| 4 | 2 g Green Tea Foam | 0.90 | 13.64 | 14.16 | |

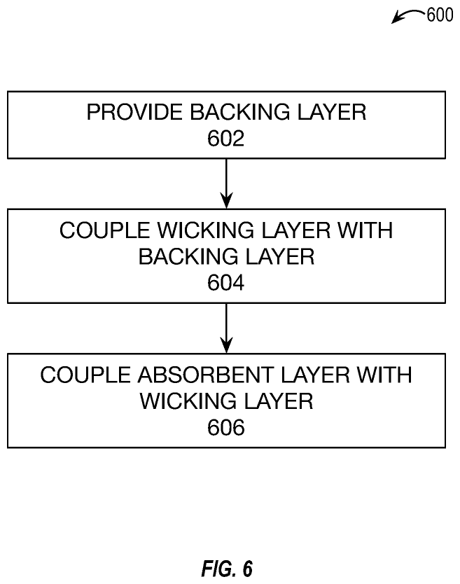

FIG. 6 illustrates an example method to form the wound dressing 110 illustrated in FIG. 1. The method 600 can include providing a backing layer (BLOCK 602). The method can include coupling a wicking layer with the backing layer (BLOCK 604). The method 660 can include coupling an absorbent layer with the wicking layer (BLOCK 606).

As set forth above, the method 600 can include providing a backing layer (BLOCK 602). The backing layer can include a first environment-facing side and a first wound-facing side. The backing layer can be liquid impermeable and vapor permeable. The backing layer can be a thin layer of polyurethane film. For example, the backing layer can include the polyurethane film ESTANE 5714F. The backing layer can include poly alkoxyalkyl acrylates and methacrylates. In some implementations, the backing layer includes a continuous layer of a high-density blocked polyurethane foam that is predominantly closed-cell.

The method 600 can include coupling a wicking layer with the backing layer (BLOCK 604). The wicking layer can be made of any material suitable for providing a wicking function to enable fluid transfer between different areas of the absorbent layer. In some implementations, the wicking layer can include rayon or other suitable wicking fabric. Several examples of wicking materials that can be used in wicking layer are described in detail in U.S. patent application Ser. No. 13/009,238 filed Jan. 19, 2011, U.S. Pat. No. 6,936,037 filed Apr. 8, 2003, and U.S. Pat. No. 9,456,930 filed Mar. 17, 2014. The entire disclosure of each of these patents and patent applications is incorporated by reference herein.

In some implementations, the wicking layer can be coupled with the backing layer by applying an adhesive to the wound-facing side of the backing layer. The application of the adhesive can be patterned to enable the passage of water vapor through the backing layer. The adhesive may include a continuous moisture vapor transmitting, pressure-sensitive adhesive layer of the type, conventionally used for island-type wound dressings (e.g., a polyurethane-based pressure sensitive adhesive). One example of an adhesive which can be used is a pressure sensitive adhesive based on acrylate ester copolymers, polyvinyl ethyl ether and polyurethane. In some implementations, the wicking layer is not coupled with the backing layer with an adhesive. Rather, the wicking layer can be positioned between the absorbent layer and the backing layer, which are coupled together.

The method 600 can include coupling an absorbent layer with the wicking layer (BLOCK 606). The absorbent layer can be a foam layer that includes about 1% and about 10%, between about 1% and about 8%, between about 1% and about 5%, between about 1% and about 4%, between about 1% and about 3%, or between about 1% and about 2% green tea by weight. The absorbent layer may include a polyurethane foam and one or more antioxidants, such as green tea. In some implementations, the absorbent layer can include a flexible plasticized hydrophilic polymer matrix having an internal cellular structure. The absorbent layer can be formed by adding green tea to one of the example hydrophilic foams in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. A wound dressing comprising:
a backing layer having a first environment-facing side and a first wound-facing side, wherein the backing layer is liquid impermeable and vapor permeable;
a wicking layer having a second environment-facing side and a second wound-facing side, wherein the second environment-facing side of the wicking layer is coupled with the first wound-facing side of the backing layer; and
an absorbent layer comprising between 1% and 10% green tea by weight, the absorbent layer having a third wound-facing side and a third environment-facing side, wherein the third environment-facing side of the absorbent layer is coupled with the second wound-facing side of the absorbent layer.

2. The wound dressing of claim 1, wherein the absorbent layer comprises between 1% and 5% green tea by weight.

3. The wound dressing of claim 1, wherein the absorbent layer comprises a hydropolymer.

4. The wound dressing of claim 1, wherein the absorbent layer comprises between 10% and 90% polyurethane by weight.

5. The wound dressing of claim 1, wherein the green tea is configured to increase a pore size of a plurality of pores in the absorbent layer.

6. The wound dressing of claim 1, wherein the green tea is configured to increase an absorbency of the absorbent layer.

7. The wound dressing of claim 1, wherein the green tea is configured to increase a rate of cellular proliferation.

8. The wound dressing of claim 1, wherein the wicking layer comprises rayon.

9. A method to form a wound dressing, comprising:
providing a backing layer having a first environment-facing side and a first wound-facing side, wherein the backing layer is liquid impermeable and vapor permeable;
coupling a second environment-facing side of a wicking layer with the first wound-facing side of the backing layer; and
coupling a third environment-facing side of an absorbent layer with the second wound-facing side of the wicking layer, wherein the absorbent layer comprises between 1% and 10% green tea by weight.

10. The method of claim 9, wherein the absorbent layer comprises between 1% and 5% green tea by weight.

11. The method of claim 9, wherein the absorbent layer comprises a hydropolymer.

12. The method of claim 9, wherein the absorbent layer comprises between 10% and 90% polyurethane by weight.

13. The method of claim 9, wherein the green tea is configured to increase a pore size of a plurality of pores in the absorbent layer.

14. The method of claim 9, wherein the green tea is configured to increase an absorbency of the absorbent layer.

15. The method of claim 9, wherein the green tea is configured to increase a rate of cellular proliferation.

16. The method of claim 9, wherein the wicking layer comprises rayon.
